(19) United States
(12) Patent Application Publication
(10) Pub. No.: US 2023/0416289 A1
(43) Pub. Date: Dec. 28, 2023

(54) METHODS FOR ELECTROREDUCTIVE LIGNIN DEPOLYMERIZATION

(71) Applicant: BOARD OF TRUSTEES OF MICHIGAN STATE UNIVERSITY, East Lansing, MI (US)

(72) Inventors: Eric Linke Hegg, East Lansing, MI (US); James E. Jackson, Haslett, MI (US); Zhen Fang, East Lansing, MI (US)

(21) Appl. No.: 18/213,429

(22) Filed: Jun. 23, 2023

Related U.S. Application Data
(60) Provisional application No. 63/354,918, filed on Jun. 23, 2022.

(51) Int. Cl. C07G 1/00; C07C 37/54; C25B 3/25

(52) U.S. Cl. CPC C07G 1/00; C07C 37/54; C25B 3/25

(57) ABSTRACT

The disclosure relates to a method for depolymerizing lignin. Prior to depolymerization, a lignin compound such as an isolated lignin with little or no crosslinking is oxidized. Lignin depolymerization includes electrochemically reducing the oxidized lignin compound by application of an electrical current through a reaction medium containing the oxidized lignin compound therein. The reaction medium can be an aqueous electrolyte medium including water, a lower alkanol solvent, an electrolyte, and a base or other pH-adjusting agent to maintain the reaction medium at an alkaline pH during electroreductive cleavage.

### CROSS REFERENCE TO RELATED APPLICATION

This application claims the benefit under 35 U.S.C. § 119(e) of U.S. Provisional Patent Application No. 63/354,918, filed Jun. 23, 2022, the entire disclosure of which is incorporated herein by reference.

### STATEMENT OF GOVERNMENT INTEREST

This invention was made with government support under DE-SC0018409 awarded by the U.S. Department of Energy. The government has certain rights in the invention.

### FIELD OF THE DISCLOSURE

The disclosure relates to methods for electrochemically depolymerizing lignin to form a reduced molecular weight lignin product with a high yield of monomeric lignin depolymerization products or residues. Lignin depolymerization products such as aromatics, alcohols, aldehydes, and ketones can be used as feedstocks for bio-renewable fuels and bio-renewable chemicals. Current techniques for lignin degradation include pyrolysis, acid- or base-catalyzed depolymerization, oxidation, gasification, enzyme-catalyzed degradation, and thio-assisted reductive electrolytic cleavage.

### BACKGROUND

The energy problem the world is faced with today stems from the number of kilowatt-hours consumed per person coupled with the rising population and the decline of fossil fuel resources supplying the kilowatt energy. Globally, 39% of the energy consumed is in the form of electric power while 27% is used for transportation, separating the energy problem into two classes: service energy and mobile energy, both of which have in the past been supplied by fossil sources. The service energy, primarily heat, refrigeration, and light, is provided by electricity. The traditionally fossil sources of this energy can be and are being replaced by renewable energies in the form of wind, solar, photovoltaics, etc. The second group, the mobile energy, represents the liquid fuels used to power trains, planes, automobiles, ships, etc. This mobile energy from energy-rich liquid fuels cannot be as easily replaced by non-liquid forms of renewable sources as the service energy group.

To tackle this liquid energy problem, research has turned to plants. Just as fossilized plants have given rise to the petroleum industry, current vegetative growth in the form of lignocellulosic biomass has given rise to the liquid-biofuel industry. It is here where research has reached a bottleneck in affordable and efficient means to turn plants into liquid fuels. In order to reach energy consumption demands, the whole plants must be utilized. Currently, in lignocellulosic biomass processing, cellulose and hemicellulose are the typical desired products due to their ability to be fermented into ethanol for production of fuel, paper, rayon, cellulose acetate, etc. However, over 50% of the plant remains unprocessed including sugar monomers that currently cannot be easily converted to ethanol, and lignin, an aromatic polymer that gives plants their structure. Current research aims to engineer microbes for digestion of non-digestible sugars, but that still leaves lignin, which contains roughly 50% of the plant's energy and carbon content.

Lignin is a complex organic polymer structure that is comprised of various aromatic subunits. These building blocks are covalently connected, most frequently by ether linkages which can comprise two-thirds or greater of the total linkages. The complexity of lignin is due to its linking mainly via six different chemical linking motifs. One illustration of the linked nature of lignin is shown in FIG. 1A.

The dominant form of the linkages in lignin is the β-O-4 ether linkage, which accounts for approximately 43-50% of lignin linkages in softwood to up to 70% in hybrid poplar. The remaining linkages are divided among a variety of carbon-carbon and ether (carbon-oxygen) bonds, depending on the lignin source.

To process and break down lignin for uses in energy or value-added products, current techniques include pyrolysis, base or acid catalyzed depolymerization, oxidation, and gasification, for example. Despite the significant advances that have been made, a simple and inexpensive process that produces few side reactions is still needed to make lignin depolymerization cost effective. One area of research in particular is the use of enzymes to catalyze the degradation of lignin. Enzymatic processes, however, are generally costly and slow.

More efficient, affordable, green methods of lignin deconstruction into its building blocks are needed to yield commercially viable chemical compounds such as aromatics, alcohols, aldehydes, etc., and thereby to fully utilize plant energy as a means of replacing fossil fuels for use in chemicals and liquid fuels.

### SUMMARY

In an aspect, the disclosure relates to a method for depolymerizing lignin, the method comprising: oxidizing a lignin compound to form an oxidized lignin compound; and electrochemically reducing the oxidized lignin compound to depolymerize the oxidized lignin compound and to form a depolymerized lignin product having a reduced molecular weight relative to the (oxidized) lignin compound prior to electrochemical reduction. The lignin compound or material can be isolated lignin obtained from biomass having already undergone Cu-AHP, GVL, or other pretreatment. An advantage of the disclosed process is that sequential lignin oxidation followed by electrochemical reduction is able to substantially depolymerize the oxidized lignin, and not simply reduce the oxidized lignin and re-form the original lignin compound. The reaction product thus includes a substantial fraction of lignin monomers and/or short lignin oligomers that are suitable feed materials for subsequent upgrading pathways.

Various refinements and embodiments of the disclosed method are possible.

In a refinement, oxidizing the lignin compound comprises performing a chemical (or reagent-based) oxidation on the lignin compound. A chemical or reagent-based oxidation as generally known in the art can be used to pre-oxidize a lignin material or compound (e.g., isolated lignin) prior to electrochemical reduction to cleave lignin linkages and form the depolymerized lignin product. The oxidation process can selectively oxidize the hydroxy group at the α-position in a β-O-4 lignin linkage to a carbonyl, for example while avoiding oxidation of the hydroxy group at the γ-position in a β-O-4 lignin linkage and avoiding crosslinking of the lignin compound. Such conversion to a corresponding carbonyl or ketone group in the β-O-4 linkage facilitates subsequent cleavage and depolymerization via electrochemical reduction. A suitable process for such oxidation includes heating a mixture of lignin, 2,3-dichloro-5,6-dicyano-1,4-benzoquinone (DDQ), and t-butyl nitrite (t-BuONO) in a suitable solvent for a sufficient time to effect conversion of the α-hydroxyl groups in the lignin. Other similar chemical oxidation processes for the selective oxidation of the hydroxy group at the α-position in a β-O-4 lignin linkage are generally known in the art, for example those using 2,2,6,6-tetramethylpiperidine 1-oxyl (TEMPO) or iron-based oxidants. In embodiments, all or substantially all (e.g., at least 80, 85 90, 95, 98, or 99% and/or up to 95, 98, 99, or 100%) of the α-hydroxyl groups can be oxidized using an oxidation process (e.g., a chemical oxidation process), which typically does not substantially alter the molecular weight of the lignin. The pre-oxidation process is suitably performed in a different reaction vessel as the subsequent electrochemical reduction. In some embodiments, the pre-oxidation process (e.g., chemical, anodic, or otherwise) can be performed in the same reaction vessel as the subsequent electrochemical reduction.

In a refinement, oxidizing the lignin compound comprises performing anodic oxidation. An electrochemical or otherwise anodic oxidation process can be performed to selectively oxidize the hydroxy group at the α-position in a β-O-4 lignin linkage to a carbonyl (i.e., with little to no oxidation of the hydroxy group at the γ-position in a β-O-4 lignin linkage and/or with little to no crosslinking of the lignin compound). Such selective oxidation of the lignin compound can be performed by application of an anodic potential to a reaction mixture including lignin dissolved in a basic (e.g., pH of at least 8, 9, or 10 and/or up to 11, 12, or 14) solvent system, for example including one or more protic solvents (e.g., water, methanol, ethanol, n-propanol, iso-propanol, n-butanol, iso-butanol, tert-butanol, acetic acid, formic acid, etc.), one or more electrolytes as described for the electrochemical reduction, and optionally one or more aprotic solvents. In some embodiments, the reaction mixture can further include an electron transfer agent. The anodic oxidation is suitably a 2e⁻ oxidation process (e.g., substantially avoiding 1e⁻ oxidation that could lead to undesirable crosslinking of phenolic moieties in the lignin compound), which is particularly useful to provide a net neutral overall process in combination with the 2e⁻ reduction process associated with electrochemical reduction of the oxidized lignin compound. For example, anodic oxidation could be performed in the anode compartment or cell of a divided electrochemical cell, and the oxidized lignin compound can then be transferred to the cathode compartment or cell of the divided electrochemical cell for electrochemical reduction and depolymerization.

In another aspect, the disclosure relates to a method for depolymerizing lignin, the method comprising: providing an oxidized lignin compound; and electrochemically reducing the oxidized lignin compound to depolymerize the oxidized lignin compound and to form a depolymerized lignin product having a reduced molecular weight relative to the lignin compound prior to electrochemical reduction, wherein the oxidized lignin compound comprises a water- or acid-soluble lignin fraction obtained from one or more biomass pretreatment methods. The lignin compound or material can be isolated lignin obtained from biomass having already undergone Cu-AHP, GVL, or other pretreatment, which can introduce at least some degree of oxidation to the lignin. An advantage of the method according to this aspect is that a lignin compound or material that is susceptible to depolymerization via electrochemical reduction can be obtained without employing a separate lignin oxidation step (e.g., no separate chemical oxidation and/or anodic oxidation is performed). It is contemplated that a lignin fraction obtained from one or more biomass pretreatment methods can be a partially oxidized lignin fraction that is susceptible to depolymerization by electrochemical reduction, without performing a separate lignin oxidation step. A further advantage of the disclosed process is that electrochemical reduction is able to substantially depolymerize the oxidized or partially oxidized lignin, and not simply reduce the oxidized or partially oxidized lignin and re-form the original lignin compound. The reaction product thus includes a substantial fraction of lignin monomers and/or short lignin oligomers that are suitable feed materials for subsequent upgrading pathways. This method can be integrated into a value added process in which (i) pretreated lignin is separated into a water-/acid-soluble fraction and an insoluble fraction (e.g., remainder/residue after extracting or separating the water-/acid-soluble fraction, such as via acid-induced precipitation of the insoluble lignin fraction), (ii) the water-/acid-soluble fraction of lignin is electrochemically reduced and depolymerized according to the disclosure, and (iii) the insoluble lignin fraction can be further processed/used without electrochemical or other depolymerization, such as in the formulation of an adhesive (e.g., polyurethane or epoxy adhesive with the insoluble lignin as a partial or full replacement for conventional polyol adhesive components). Alternatively, this method can be integrated into a value added process in which (i) pretreated lignin is electrochemically reduced and depolymerized without separation (e.g., as provided by/resulting from the pretreatment process), which will preferentially depolymerize the oxidized lignin portion of the pretreated lignin, (ii) the electrochemical reaction product is then separated into a water-/acid-soluble fraction and an insoluble fraction (e.g., via acid addition to precipitate the insoluble lignin fraction while retaining the lignin monomers and/or short lignin oligomers resulting from depolymerization in solution), and (iii) the insoluble lignin fraction can be further processed/used without electrochemical or other depolymerization, such as in the formulation of an adhesive as described above. In general for the various disclosed embodiments and as described below, the electrochemical depolymerization reaction is typically performed in an alkaline/high-pH environment to solubilize the lignin reactant. The water-/acid-soluble fraction and insoluble fraction can be separated from each other (e.g., after depolymerization) by addition of an acid to the product medium, which will precipitate insoluble lignin (e.g., non-oxidized or otherwise larger lignin chains that were not substantially depolymerized), but which will retain the lignin monomers and/or short lignin oligomers resulting from depolymerization in the acidified aqueous product medium. The acid used to perform the fractionation and obtain the water-/acid-soluble fraction is not particularly limited, although mineral acids such as sulfuric acid, nitric acid, and hydrochloric acid can be used. The pH of the acidified aqueous product medium is not particularly limited, although a target pH value of about 2 (e.g., 1 to 4, 1.5 to 3, or 1.5 to 2.5) is often sufficient to precipitate a substantial portion or essentially all of the insoluble lignin (e.g., where relative degree of precipitation increases with lower pH).

In a refinement, the lignin compound comprises an isolated lignin obtained from one or more biomass pretreatment methods selected from the group consisting of a γ-valerolactone/sulfuric acid (GVL) pretreatment and a copper-catalyzed alkaline hydrogen peroxide (Cu-AHP) pretreatment. For example, the isolated lignin can be obtained from biomass having been subjected to a single pretreatment/isolation method, a mixture of isolated lignins obtained from biomass subjected to different pretreatment methods, etc. In embodiments, all of the lignin is an isolated lignin obtained from GVL, Cu-AHP, or other pretreatment that does not introduce a substantial amount of crosslinks. In other embodiments, the disclosed method can be applied to kraft, soda, organosolv, etc. lignin to obtain at least some depolymerization benefit, even if the degree of depolymerization can be lessened due to the introduction of crosslinks from kraft, soda, organosolv, or other biomass pretreatments.

In a refinement, the lignin compound comprises a phenolic polymer comprising at least one of β-O-4 ether linkages, α-O-4 ether linkages, 4-O-5 ether linkages, β-β carbon-carbon linkages, β-5 carbon-carbon linkages, and 5-5 carbon-carbon linkages between phenolic monomer units in the phenolic polymer.

In a refinement, the depolymerized lignin product has 50% or less linkages between phenolic monomer units in the phenolic polymer relative to the (oxidized) lignin compound prior to electrochemical reduction.

In a refinement, the depolymerized lignin product has a number- or weight-average molecular weight of 50% or less relative to the number- or weight-average molecular weight of the (oxidized) lignin compound prior to electrochemical reduction.

In a refinement, the (oxidized) lignin compound prior to electrochemical reduction has a number-average molecular weight of at least 400 g/mol. In a further refinement, the depolymerized lignin product has a number-average molecular weight of 50% or less relative to the number-average molecular weight of the (oxidized) lignin compound prior to electrochemical reduction.

In a refinement, the (oxidized) lignin compound prior to reacting has a weight-average molecular weight of at least 10000 g/mol. In a further refinement, the depolymerized lignin product has a weight-average molecular weight of 50% or less relative to the weight-average molecular weight of the (oxidized) lignin compound prior to electrochemical reduction.

In a refinement, the depolymerized lignin product comprises at least one of a lignin monomer unit and a lignin oligomer thereof, the lignin monomer unit comprising an aromatic group with one or more substituents selected from the group consisting of alcohol groups, ether groups, aldehyde groups, ketone groups, alkyl groups, carboxyl groups, and combinations thereof. In a further refinement, the lignin monomer unit is selected from the group consisting of syringic acid, vanillin, para-hydroxybenzoic acid (p-OH benzoic acid), acetosyringone, propiosyringone, phenol, cresol, guaiacol, 4-ethyl-guaiacol, eugenol, isoeugenol, methoxyeugenol, syringol, and combinations thereof.

In a refinement, the depolymerized lignin product comprises lignin monomer units in an amount of 5 wt. % to 50 wt. % relative to the (oxidized) lignin compound prior to electrochemical reduction. More generally, the depolymerized lignin product can include lignin monomer units in an amount of at least 5, 10, 15, 20, 25, 30, 40, 50, 60, 70, or 80 wt. % and/or up to 20, 35, 50, 65, 75, 85, 95, or 100 wt. % relative to the (oxidized) lignin compound prior to electrochemical reduction. In some embodiments, one or more of syringic acid, vanillin, p-OH benzoic acid, acetosyringone, and propiosyringone constitute about 50-95 wt. % of the lignin monomers, for example at least 50, 60, or 70 wt. % and/or up to 75, 85, or 95 wt. % the lignin monomers. Alternatively or additionally, the depolymerized lignin product can include lignin oligomers in an amount of at least 5, 10, 15, 20, 25, 30, 40, 50, 60, 70, or 80 wt. % and/or up to 20, 35, 50, 65, 75, 85, 95, or 100 wt. % relative to the (oxidized) lignin compound prior to electrochemical reduction. The lignin oligomers can have 2-10 repeat units (e.g., at least 2, 3, or 4 and/or up to 6, 8, or 10). Alternatively or additionally, the depolymerized lignin product can include a combined amount of lignin monomer units and lignin oligomers in an amount of at least 10, 20, 30, 40, 50, 60, 70, 80, 90, or 95 wt. % and/or up to 30, 40, 50, 65, 75, 85, 95, 98, or 100 wt. % relative to the (oxidized) lignin compound prior to electrochemical reduction.

In a refinement, an ethyl acetate-soluble fraction of the depolymerized lignin product constitutes 5 wt. % to 60 wt. % or 15 wt. % to 50 wt. % of the depolymerized lignin product, for example at least 5, 10, 15, 20, or 30 wt. % and/or up to 20, 30, 40, 50, or 60 wt. %. Alternatively or additionally, a water-soluble fraction of the depolymerized lignin product constitutes 5 wt. % to 60 wt. % or 15 wt. % to 50 wt. % of the depolymerized lignin product, for example at least 5, 10, 15, 20, or 30 wt. % and/or up to 20, 30, 40, 50, or 60 wt. %. Alternatively or additionally, combined ethyl acetate- and water-soluble fractions of the depolymerized lignin product constitute 10 wt. % to 95 wt. % or 30 wt. % to 80 wt. % of the depolymerized lignin product, for example at least 10, 20, 30, 40, 50, or 60 wt. % and/or up to 50, 60, 70, 80, 90, or 95 wt. %.

In a refinement, the method comprises electrochemically reducing the oxidized lignin compound in an aqueous electrolyte (e.g., catholyte) medium. In a further refinement, the aqueous electrolyte medium comprises: water; optionally a solvent; wherein the aqueous electrolyte medium has a pH value of at least 8 (e.g., pH of at least 8, 9, or 10 and/or up to 11, 12, or 14; resulting from the addition of a base such as NaOH, KOH, or other alkali metal hydroxides). Examples of solvents include protic solvents such as one or more of methanol, ethanol, n-propanol, iso-propanol, n-butanol, iso-butanol, tert-butanol, acetic acid, formic acid, and combinations thereof. Typically some non-water protic solvent is included to help solubilize the (oxidized) lignin compound and depolymerized lignin product components, although in some embodiments water can be substantially the only solvent. In embodiments, water can be present in the aqueous electrolyte medium in an amount of at least 10, 20, 30, 40, 50, 60, or 70 wt. % and/or up to 40, 50, 60, 70, 80, 90, 95, or 100 wt. % relative to the aqueous electrolyte medium. Alternatively or additionally, one or more solvents can be present in the aqueous electrolyte medium in an amount of at least 10, 20, 30, 40, 50, or 60 wt. % and/or up to 40, 50, 60, 70, 80, or 90 wt. % relative to the aqueous electrolyte medium. The aqueous electrolyte medium can have a pH of at least 8, 9, or 10 and/or up to 11, 12, or 14, for example resulting from the addition of a base such as NaOH, KOH, or other alkali metal hydroxide to the aqueous electrolyte medium. Examples of suitable electrolytes include NaCl, KCl, LiBF₄, other alkali metal halides or other salts. Suitable electrolyte concentrations can be about 0.01-5 M, 0.05-1 M, 0.07-0.5 M, or 0.1-0.3 M. Such concentrations are generally sufficient to promote depolymerization, although at varying rates. In a further refinement, the oxidized lignin compound is initially present (e.g., added to) the aqueous electrolyte medium in an amount of 0.1, 1, 2, 5, or 10 g/L to 2, 5, 10, 20, 50, or 100 g/L (e.g., mass of oxidized lignin compound relative to volume of aqueous electrolyte medium).

In a refinement, the method comprises electrochemically reducing the oxidized lignin compound in a cathode compartment of a divided electrochemical cell (e.g., with the aqueous electrolyte medium described above as the catholyte and reaction medium in the cathode compartment).

In a refinement, the method comprises electrochemically reducing the oxidized lignin compound in an undivided electrochemical cell.

In a refinement, the method comprises electrochemically reducing the oxidized lignin compound: (i) at a temperature in a range from 20° C. to 200° C.; (ii) at a pressure in a range of 0.5 bar to 5 bar; (iii) for a time in a range of 0.1 hr to 50 hr; and/or (iv) at a current density in a range of 1 mA/cm² to 1000 mA/cm².

In a refinement, the method comprises electrochemically reducing the oxidized lignin compound in the absence of an added thio (or sulfur-containing) compound. For example, the aqueous electrolyte medium can be free from thio or sulfur-containing compounds.

While the disclosed compounds, methods, and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### BRIEF DESCRIPTION OF THE DRAWINGS

FIG. 1A illustrates chemical structures representative of lignin, in particular a linked lignin backbone structure.

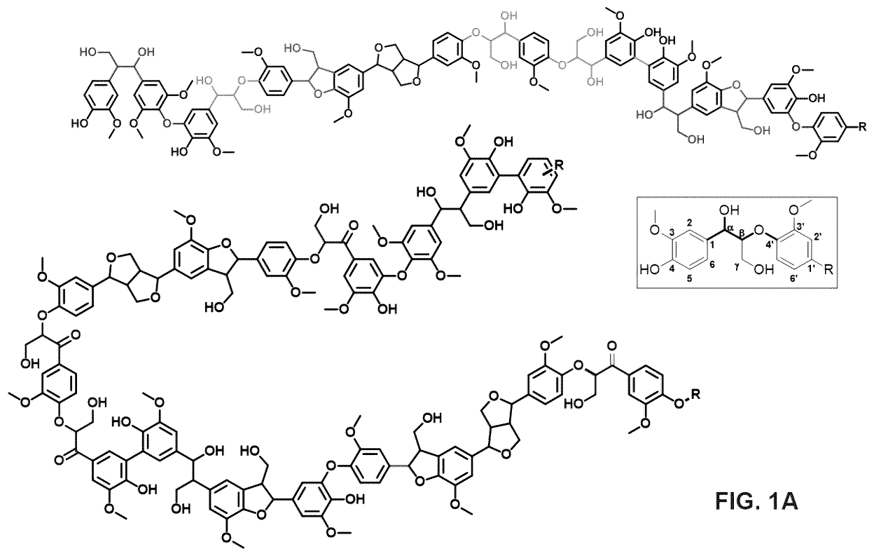

FIG. 1B illustrates chemical structures representative of lignin, in particular showing detail of the various linkages found in lignin.

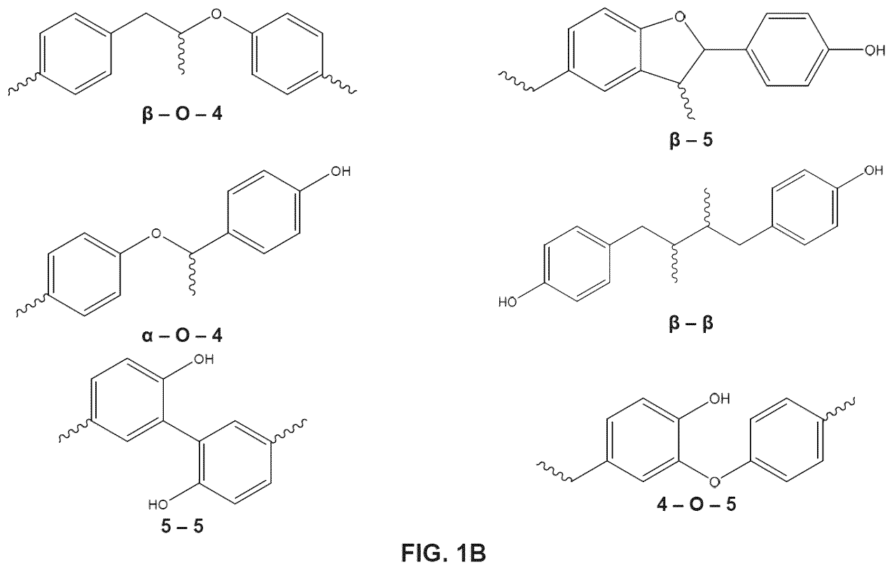

FIG. 2 shows the oxidation of a hydroxy group in a β-O-4 linkage in lignin to form a corresponding carbonyl or ketone group at the α-position.

FIG. 3 illustrates an undivided electrochemical cell 100 composed of cathode 1 and anode 2 in the same electrochemical chamber 3.

FIG. 4 illustrates a divided electrochemical cell 200 composed of cathode 6 and anode 7 in different electrochemical chambers (anode chamber 8 and cathode chamber 9) separated by an ion exchange membrane 10.

FIG. 5 illustrates a schematic of the process of electrolysis of lignin samples and isolation of EA-soluble and insoluble fractions.

FIGS. 6A and 6B show yields of reaction products and consumption of starting material for electrochemical reduction of 2-phenoxyacetophenone under various conditions.

### DETAILED DESCRIPTION

Provided herein are methods of depolymerizing lignin. In general, the methods of the disclosure include oxidizing a lignin compound to form an oxidized lignin compound, and then electrochemically reducing the oxidized lignin compound to depolymerize the oxidized lignin compound and to form a depolymerized lignin product. The depolymerized lignin product can have a substantial fraction of solubilized lignin depolymerization products (e.g., lignin monomers and/or oligomers thereof) and/or a substantially reduced molecular weight relative to the lignin compound prior to electrochemical reduction.

### Lignin Compound

Plants and their associated biomass, in general, are comprised of cellulose, hemicellulose, pectin, and lignin. Cellulose can be thought of as a crystalline glucose polymer. It is a six-carbon sugar that is a major structural component of cell walls. Hemicellulose is a mixture of five and six-carbon sugars with an amorphous structure that acts as a branching polymer that cross links around the cellulose. Pectin is a complex polysaccharide that helps bind the plant cells together. Lignin is an amorphous aromatic polymer that is made of methoxy substituted catechols. The more methoxy substitutions on the aromatic polymer, the easier the plant is to degrade. This is due to radical-generated linking between non-substituted positions on the ring making lignin a stronger polymer. Cellulose, hemicellulose, and lignin give the plant structural support. The linkages that dictate these interactions are important for the deconstruction of this feedstock. Intramolecular linkages include: hydrogen and ether bonds in cellulose; ester and ether bonds in hemicellulose; and carbon-carbon and ether bonds in lignin. Intermolecular linkages allow the lattice-like interaction and include: ether and hydrogen bonds between lignin and cellulose; ether, ester, and hydrogen bonds between lignin and hemicellulose; and hydrogen bonding between cellulose and hemicellulose. It is these stronger bonds with lignin that reduce the efficiency of lignocellulosic deconstruction.

The β-O-4 ether bond is the predominant linkage in lignin, but various linkages make up this aromatic polymer, including: β-O-4, α-O-4, β-5, 4-O-5, 5-5, and β-β, with the percentage of each varying between numerous feedstocks. These linkages have their own naming system separate from the IUPAC nomenclature. To name these linkages, numbering of the ring starts at the propyl unit, followed by numbering to the closest methoxy group; from the propyl group, the Greek alphabet is counted out. More generally, a lignin linkage connects phenolic monomers along the main lignin chain, for example including native bonds between phenolic monomers in lignin present in the original biomass as well as in isolated lignin after biomass pretreatment. These linkages or native bonds typically include bonds linking phenolic monomer units along a substantially linear backbone (e.g., β-O-4, α-O-4, β-5, 4-O-5, 5-5, and/or β-β bonds), although it is possible that some branching or other bonds (e.g., C—C bonds, whether 5-5 bonds or otherwise) can be present in the original biomass and corresponding isolated lignin. Such linkages can be contrasted with crosslinks that are introduced into the isolated lignin during biomass pretreatment and that are typically different chemical bonds from those originally present in the lignin. For example, such crosslinks can be characterized as C—C bonds introducing branching into a lignin polymeric backbone and/or linking/joining multiple polymeric backbone units together. Alternatively or additionally, the crosslinks can be characterized as monomer-linking bonds other than β-O-4, α-O-4, β-5, 4-O-5, 5-5, and/or β-β linkages (e.g., which similarly introduce branching and/or linking of multiple backbone units together). The presence of substantial crosslinks introduced from pretreatment can reduce the ability of the disclosed method to depolymerize the lignin. Accordingly, in some embodiments, a biomass pretreatment method can be selected to provide an isolated lignin with little or no additional crosslinks, for example a GVL or Cu-AHP pretreatment process as described below. The introduction of crosslinks into isolated lignin relative to the original biomass can be characterized by the relative distribution of β-O-4, α-O-4, β-5, 4-O-5, 5-5, and/or β-β linkages in the original biomass as compared to the isolated lignin, where similar or substantially close values for a given linkage between original biomass and the isolated lignin represent the introduction of little or no additional crosslinks. In embodiments, the amount of a given β-O-4, α-O-4, β-5, 4-O-5, 5-5, or β-β linkage in an isolated lignin (e.g., expressed as a number- or weight-based fraction or percentage of total inter-monomer bonds or linkages) is suitably within a window±0.1, 0.2, 0.5, 1, 2, 4, 6, 8, or 10% on an absolute or relative basis compared to the original biomass. For example, given an original biomass with about 60% β-O-4 linkages, a±10% window on an absolute basis corresponds to about 50-70% β-O-4 linkages in the corresponding isolated lignin (i.e., 60% ±10%), while a±10% window on a relative basis corresponds to about 54-66% β-O-4 linkages in the corresponding isolated lignin (i.e., 60%±(10% of 60%)).

In hybrid poplar, an important feedstock due to its fast growth and ability to grow on marginal lands, the β-O-4 bond is the major linkage, accounting for roughly 70% with the other five linkage types accounting for the remaining 30%. It is this β-aryl ether bond that has been the primary focus of lignin deconstruction.

The lignin compound depolymerized according to the methods of the disclosure is not particularly limited. The lignin compound can include a linked phenolic polymer including at least one of β-O-4 ether linkages, α-O-4 ether linkages, 4-O-5 ether linkages, β-β carbon-carbon linkages, β-5 carbon-carbon linkages, and 5-5 carbon-carbon linkages between phenolic monomer units in the phenolic polymer. In some embodiments, the lignin compound is characterized by at least 40%, 50%, or 60% and/or up to 60%, 70%, 75%, or 80% of its linkages as being β-O-4 ether linkages. In some embodiments, the lignin compound is characterized by at least 1%, 2%, 3%, or 5% and/or up to 7%, 10%, 15%, or 20% of its linkages as being α-O-4 ether linkages. In some embodiments, the lignin compound is characterized by at least 1%, 2%, 3%, or 5% and/or up to 7%, 10%, 15%, or 20% of its linkages as being β-β linkages. In some embodiments, the lignin compound is characterized by at least 0.1%, 0.2%, 0.3%, 0.5%, or 1% and/or up to 3%, 5%, 10%, or 15% of its linkages as being α-α linkages. In some embodiments, the lignin compound is characterized by at least 0.1%, 0.2%, 0.3%, 0.5%, or 1% and/or up to 1%, 2%, 3%, or 5% of its linkages as being 5-5 linkages. In some embodiments, the lignin compound is characterized by at least 0.1%, 0.2%, 0.3%, 0.5%, or 1% and/or up to 1%, 2%, 3%, or 5% of its linkages as being 4-O-5 linkages. The foregoing ranges can similarly apply to linkages present in the original biomass and/or the isolated lignin.

Furthermore, the lignin compound can be in any desired form and from any desired biomass or lignocellulosic feedstock. For example, the lignin compound can be an isolated lignin material or a lignocellulosic material in combination with cellulose and/or hemicellulose. In embodiments, the lignin compound is at least partially oxidized to include lignin ketone groups in place of native lignin alcohol groups. The lignin compound can be an untreated lignin compound or it can be mechanically and/or chemically pre-treated prior to depolymerization. In an embodiment, the lignin compound is an isolated lignin obtained from biomass using one or more pretreatment (e.g., fractionation and/or extraction) methods prior to being depolymerized according to the disclosure. Common biomass pretreatment methods known in the art include kraft, soda, organosolv, γ-valerolactone/sulfuric acid (GVL), and copper-catalyzed alkaline hydrogen peroxide (Cu-AHP) pretreatments. In embodiments, the lignin is an isolated lignin obtained from biomass pretreated with a Cu-AHP or a GVL pretreatment. Cu-AHP and GVL pretreatments are particularly suitable because they limit the introduction of additional crosslinks into the isolated lignin (i.e., in addition to the phenolic monomer linkages naturally present), which in turn promotes subsequent lignin depolymerization via the disclosed method. Other pretreatments that at least partially oxidize lignin alcohol groups to ketone groups are also suitable. In embodiments, the lignin compound includes any or all of its original carbon-oxygen-carbon ether linkages and/or carbon-carbon linkages. In embodiments where the lignin is an isolated lignin obtained from biomass pretreatment, the lignin can include a subset of its original carbon-oxygen-carbon ether linkages and/or carbon-carbon linkages.

The number-average molecular weight (Mn) of the lignin compound prior to reacting or subjecting to electroreductive depolymerization is not particularly limited. The number-average molecular weight of the lignin compound prior to reacting can be at least about 400 g/mol, about 1000 g/mol, 2000 g/mol, 5000 g/mol or about 10,000 g/mol, up to about 2000 g/mol, about 5000 g/mol, about 10,000 g/mol, about 20,000 g/mol, or about 50,000 g/mol. That is, the number-average molecular weight of the lignin compound prior to reacting can range from about 400 g/mol to about 50,000 g/mol, about 1000 g/mol to about 20,000 g/mol, about 2000 g/mol to about 10,000 g/mol, for example, about 400, 500, 600, 700, 800, 900, 1000, 1250, 1300, 1500, 1750, 2000, 2500, 3000, 3500, 4000, 4500, 5000, 5500, 6000, 6500, 7000, 7100, 7200, 7500, 8000, 8500, 9000, 10,000, 15,000, 20,000, 25,000, 30,000, 35,000, 40,000, 45,000 and 50,000 g/mol.

The weight-average molecular weight (Mw) of the lignin compound prior to reaction or being subjected to electroreductive depolymerization is not particularly limited. The weight-average molecular weight of the lignin compound prior to reacting can be at least about 10,000 g/mol, about 20,000 g/mol, about 30,000 g/mol, about 40,000 g/mol, or about 50,000 g/mol up to about 20,000 g/mol, about 50,000 g/mol, about 100,000 g/mol, about 200,000 g/mol, or about 500,000 g/mol. That is, the weight-average molecular weight of the lignin compound prior to reacting can range from about 10,000 g/mol to about 500,000 g/mol, about 20,000 g/mol to about 200,000 g/mol, about 30,000 g/mol to about 100,000 g/mol, or about 40,000 g/mol to about 50,000 g/mol, for example about 10,000, 11,000, 15,000, 20,000, 30,000, 40,000, 45,000, 46,000, 50,000 52,000, 55,000, 56,000, 60,000, 65,000, 70,000, 80,000, 90,000, 100,000, 125,000, 200,000, 250,000, 300,000, 350,000, 400,000, 450,000, and 500,000 g/mol.

### Lignin Oxidation

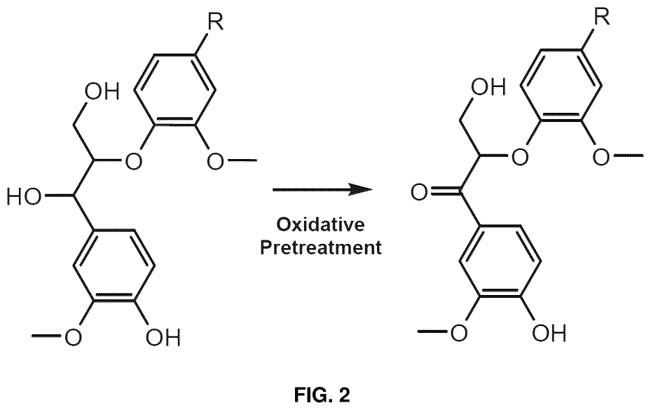

The method according to the disclosure includes oxidizing the lignin compound to form an oxidized lignin compound prior to electrochemical reduction and depolymerization. As illustrated in FIG. 2, oxidation of lignin suitably includes selective oxidation of the hydroxy group at the α-position in a β-O-4 lignin linkage to a carbonyl. Such conversion to a corresponding ketone group in the β-O-4 linkage facilitates subsequent cleavage and depolymerization via electrochemical reduction.

### Lignin Depolymerization Reaction

Lignin depolymerization according to the disclosure generally includes electrochemically reducing the oxidized lignin compound by application of an electrical current through a reaction medium containing the oxidized lignin compound therein. In embodiments, the reaction medium can be an aqueous electrolyte (e.g., catholyte) medium including water, a lower alkanol solvent, an electrolyte, and a base or other pH-adjusting agent to maintain the reaction medium at an alkaline pH during electroreductive cleavage.

In embodiments, electrochemical reduction and depolymerization of lignin (or pre-oxidized lignin) can be performed in a suitable electrochemical reactor or cell, for example in an undivided electrochemical cell or a divided electrochemical cell.

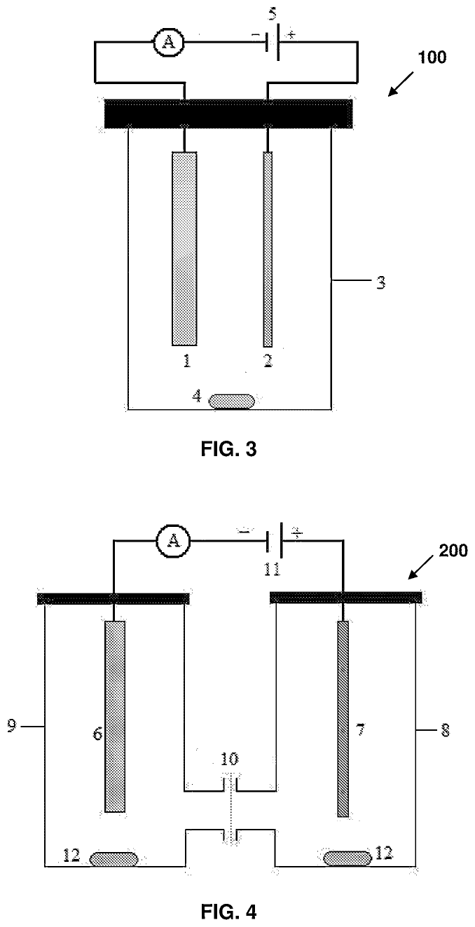

An example of an undivided cell 100 is shown in FIG. 3, where the cathode 1 and the anode 2 are in the same electrochemical chamber 3. Lignin (or pre-oxidized lignin) is added into the electrochemical chamber 3 for the electroreductive depolymerization. Power supply 5 provides electrons to cathode 1 for the reduction reaction, while the anode 2 releases electrons to the power supply. Stirring is used to enhance mass transfer with a magnetic stirring bar 4. An ammeter (not shown) is used to measure the current.

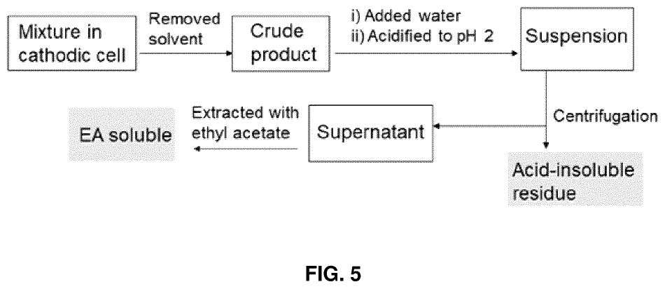

An example of a divided cell 200 is shown in FIG. 4, which includes a cathode 6 and an anode 7 in different electrochemical chambers (anode chamber 8 and cathode chamber 9) separated by an ion exchange membrane 10. NAFION membranes, such as NAFION 115 and NAFION 117, are suitable (available from Dupont), but other membranes can be used as well. Lignin (or pre-oxidized lignin) is added into the cathode chamber 9 and aqueous solution with electrolytes is put into the anode chamber 8. Power supply 11 provides the electrons to the cathode 6. Magnetic stirring bar 12 is used to mix the solution to enhance mass transfer. An ammeter (not shown) is used to measure the current.

Suitable anode materials include platinum, stainless steel, graphite, etc. The anode can be made of bulk materials including platinum wire, platinum mesh, platinized titanium mesh, stainless steel wire, stainless steel mesh and graphite rod. Precious metals supported on carbon and/or a high surface area material, such as platinum on activated carbon cloth, can also be used as an anode. Suitable cathode materials include electron-transfer materials such as lead, nickel, and carbon, for example reticulated vitreous carbon (RVC).

In embodiments, the electrochemical reduction is performed in the absence of any catalyst materials, i.e., materials that would catalyze the electrochemical reduction. For example, the electrochemical reduction can be performed in a reaction medium that does not include a dispersed heterogeneous catalyst or a dissolved homogeneous catalyst. Furthermore, the electrochemical reduction can be performed using electrodes (i.e., anode, cathode, or both) that contain no catalyst materials, either made therefrom or deposited thereon.

The reaction conditions of the depolymerization reaction are not particularly limited.

In embodiments, electrochemical reduction of the oxidized lignin compound is performed at a temperature ranging from about 0° C. to about 200° C., about 25° C. to about 150° C., about 30° C. to about 120° C., about 50° C. to about 100° C., or about 70° C. to about 90° C., for example about 0, 10, 20, 25, 30, 35, 40, 50, 60, 70, 75, 80, 90, 100, 110, 120, 130, 140, 150, 160, 170, 180, 190, or 200° C. The reaction temperature is suitably chosen to be at or below the boiling point temperature of any solvent medium. In embodiments, electrochemical reduction of the oxidized lignin compound is performed at a pressure ranging from about 0.5 bar to about 50 bar, about 0.6 bar to about 10 bar, about 0.8 bar to about 5 bar, about 0.8 bar to about 2 bar, or about 0.9 bar to about 15 bar, for at least 0.5, 0.6, 0.8, 0.9, 1, 1.5, 2, or 5 bar and/or up to 1.2, 1.5, 2, 5, 10, 20, 30, or 50 bar. In embodiments, electrochemical reduction of the oxidized lignin compound is performed at a current density in a range of 1 mA/cm² to 1000 mA/cm², for example at least 1, 2, 5, 10, 20, 50, or 100 mA/cm² and/or up to 10, 20, 50, 100, 200, 500, or 1000 mA/cm².

Electrochemical reduction of the oxidized lignin compound can be performed in a batch, semi-batch, or continuous process. The reaction time in a batch reaction system or residence time in a continuous reaction system can be suitably selected to achieve a suitable conversion and/or yield of the lignin depolymerization product. That is, reaction times may range from about 0.1 hour to about 48 hours. For example, reaction times can be at least 0.1, 0.2, 0.3, 0.5, 0.7, 1, 1.5, 2, 4, 6, or 12 hr and/or up to 0.4, 0.6, 0.8, 1, 1.2, 1.6, 2, 4, 8, 16, 24, 36, 48, or 50 hr.

The pH of the reaction medium is suitably alkaline. In embodiments, electrochemical reduction of the oxidized lignin compound is performed at a pH value of at least 8, for example at a value of at least 8, 9, 10, 11, or 12 and/or up to 10, 11, 12, 13, or 14. The pH value as described herein can apply to the pH of the reaction medium initially, during electrochemical reduction, and/or at the end of electrochemical reduction.

The method of the disclosure provides a depolymerized lignin product.

In embodiments, the depolymerized lignin product has fewer linkages between phenolic monomer units in the phenolic polymer relative to the (oxidized) lignin compound prior to reacting. The depolymerized lignin product can have about 50% or less, or 60% or less, linkages between phenolic monomer units in the phenolic polymer relative to the lignin compound prior to reacting. For example, the depolymerized reaction product can have from less than about 1% to up to about 50% or 60%, less than about 2% to about 40%, less than about 5% to about 30%, or less than about 10% to about 20% linkages, for example about 1%, 2%, 5%, 10%, 15%, 20%, 25%, 30%, 35%, 40%, 45%, 50%, 55%, or 60% linkages between phenolic monomer units in the phenolic polymer relative to the (oxidized) lignin compound prior to reacting.

The term “linkage,” as used herein, can refer to all types of linkages in the starting material, for example, β-O-4, α-O-4, β-5, 4-O-5, 5-5, and/or β-β. Accordingly, the percentage of linkages remaining in the depolymerized lignin product can be expressed relative to all types of linkages in the starting lignin material. Similarly, the percentage of linkages remaining in the depolymerized lignin product can be expressed relative to a particular type of linkage in the starting lignin material. For example, in some embodiments, the β-O-4 ether linkages are cleaved in the depolymerized lignin product and are reduced relative to the (oxidized) lignin compound prior to reacting, providing a depolymerized lignin product having fewer β-O-4 ether linkages between phenolic monomer units in the phenolic polymer relative to the (oxidized) lignin compound prior to reacting. That is, the depolymerized lignin product can have from less than about 1% to up to about 50% or 60%, less than about 2% to about 40%, less than about 5% to about 30%, or less than about 10% to about 20% β-O-4 ether linkages, for example about 1%, 2%, 5%, 10%, 15%, 20%, 25%, 30%, 35%, 40%, 45%, 50%, 55%, or 60% β-O-4 ether linkages, relative to the starting lignin material.

In general, the depolymerized lignin product has a molecular weight less than that of the (oxidized) lignin compound prior to reacting. In embodiments, the depolymerized lignin product has a molecular weight of 50% or less, or 60% or less, relative to the molecular weight of the (oxidized) lignin compound prior to reacting. For example, the depolymerized lignin product can have a molecular weight from about 1% to about 50% or 60%, about 2% to about 40%, about 5% to about 30%, or about 10% to about 20%, for example about 1%, 2%, 5%, 10%, 15%, 20%, 25%, 30%, 35%, 40%, 45%, 50%, 55%, or 60% relative to the (oxidized) lignin compound prior to reacting. As used herein, relative molecular weight reduction by depolymerization can be expressed on any suitable molecular weight basis, such as, for example, number-average molecular weight, or weight-average molecular weight.

For example, in embodiments, the depolymerized lignin product has a number-average molecular weight of 50% or less, or 60% or less, relative to the number-average molecular weight of the (oxidized) lignin compound prior to reacting. That is, the depolymerized lignin product has a number-average molecular weight of from about 1% to about 50% or 60%, about 2% to about 40%, about 5% to about 30%, or about 10% to about 20%, for example about 1%, 2%, 5%, 10%, 15%, 20%, 25%, 30%, 35%, 40%, 45%, 50%, 55%, or 60%, relative to the number-average molecular weight of the (oxidized) lignin compound prior to reacting.

Similarly, in embodiments, the depolymerized lignin product has a weight-average molecular weight of 50% or less, or 60% or less, relative to the weight-average molecular weight of the (oxidized) lignin compound prior to reacting. That is, the depolymerized lignin product has a weight-average molecular weight of from about 1% to about 50% or 60%, about 2% to about 40%, about 5% to about 30%, or about 10% to about 20%, for example about 1%, 2%, 5%, 10%, 15%, 20%, 25%, 30%, 35%, 40%, 45%, 50%, 55%, or 60%, relative to the weight-average molecular weight of the (oxidized) lignin compound prior to reacting.

In embodiments, the depolymerized lignin product includes at least one of a lignin monomer unit and a lignin oligomer thereof, the lignin monomer unit including an aromatic group with one or more substituents. As used herein, “an aromatic group with one or more substituents” refers to a C₆ mono- or poly-substituted aromatic ring including substituents having 0, 1, 2, 3, 4, or 5 carbon atoms. The particular substituent on the aromatic ring is not particularly limited. For example, suitable substituents include, but are not limited to, alcohol groups, ether groups, aldehyde groups, ketone groups, alkyl groups, carboxyl groups, alkoxy groups, and combinations thereof. For example, a hydroxyl, —OH, substituent contains 0 carbon atoms, while other substituents, such as a ketone or an aldehyde group, contain at least 1 and up to 5 carbon atoms.

The lignin monomer is not particularly limited and can be selected from any monomer that is suitable to form a lignin oligomer or a lignin polymer. Examples of lignin monomers include, but are not limited to, syringic acid, vanillin, p-OH benzoic acid, acetosyringone, propiosyringone, phenol, cresol, guaiacol, 4-ethyl-guaiacol, eugenol, isoeugenol, methoxyeugenol, syringol, acetophenone, and combinations thereof. The lignin oligomer can include any suitable mixture or arrangement of lignin monomers.

The lignin oligomer can contain from about 2 to about 50 or 75 or 100, about 3 to about 30, about 5 to about 20, or about 10 to about 15 lignin monomer units, for example, about 2, 3, 5, 10, 15, 20, 25, 30, 35, 40, 45, 50, 75, or 100 lignin monomer units. The monomer units of the lignin oligomer are not particularly limited, and can include a plurality or distribution of different lignin monomer units and lignin oligomers with differing types and numbers of monomer units.

### EXAMPLES

The following examples illustrate the methods according to the disclosure.

Lignin samples from four common pretreatment methods (kraft, soda, γ-valerolactone/sulfuric acid (GVL), and copper-catalyzed alkaline hydrogen peroxide (Cu-AHP) were used. These lignins are referred to herein as Kraft, Soda, GVL, and Cu-AHP, respectively, and their oxidized forms are referred to herein as Kraft^{ox}, Soda^{ox}, GVL^{ox}, and Cu-AHP^{ox}, respectively. The oxidized lignins are collectively referred to herein as lignin^{ox}.

Analytical grade syringic acid, vanillin, p-OH benzoic acid, acetosyringone, and propiosyringone were purchased from Sigma-Aldrich. Kraft and soda lignin were provided by Suzano (São Paulo, Brazil) and Green Value (Orbe, Switzerland), respectively. GVL lignin from hybrid poplar [Populus nigra L.×P. maximowiczii A Henry (NM6)] was obtained from the Great Lakes Bioenergy Research Center (University of Wisconsin—Madison, USA), and Cu-AHP lignin from hybrid poplar [P. nigra var. charkoviensis×caudina cv. NE-19] was produced via copper-catalyzed alkaline hydrogen peroxide pretreatment using 1 mM of copper(II) sulfate and 2 mM of 2,2′-bipyridine, as described in Bhalla, A., et al., Biotechnol. Biofuels 2016, 9, 34, the entire contents of which are hereby incorporated by reference.

Lignin oxidation. DDQ-catalyzed oxidation of lignins was performed according to a known procedure. Briefly, a mixture of 3 g of lignin, DDQ (0.3 g; 1.3 mmol), and t-BuONO (0.3 g; 2.9 mmol) was dissolved in 42 mL of a 60/40 (v/v) mixture of 1,2-dimethoxyethane/2-ethoxyethanol, followed by heating at 80° C. under an oxygen atmosphere for 14 h. The oxidized lignin product was precipitated by the addition of 200 mL of diethyl ether, isolated by vacuum filtration, washed with additional diethyl ether (100 mL), and dried in vacuo.

Cathodic electrolysis of 2-phenoxyacetophenone. Electroreduction of 2-phenoxyacetophenone was used as a model reaction to establish reaction conditions, in particular the choice of solvent, for lignin electrolysis. Electroreductions of 2-phenoxyacetophenone were conducted at room temperature for 2 h in an H-type electrochemical cell, separated by a Nafion^{™} 117 membrane, wherein a platinum wire in 20 mL of pH 8 phosphate buffer was placed in the anode half-cell and reticulated vitreous carbon (RVC, 2 cm² immersed) was employed as the cathode. Both half-cells were equipped with magnetic stir bars and were vigorously stirred during the reaction. The start of the reaction (t=0) was defined as the time when the substrate (2-phenoxyacetophenone) was added to the cell. Briefly, 20 mg of 2-phenoxyacetophenone was added directly to the cathodic cell and dissolved with stirring in a mixture of 10 mL of methanol and 10 mL of an aqueous electrolyte solution. Once the 2-phenoxyacetophenone was completely dissolved, 20 mA electric current was applied, and the current was maintained for the duration of the reaction. The electroreduction of 2-phenoxyacetophenone generates phenol, acetophenone, and a side product, pinacol:

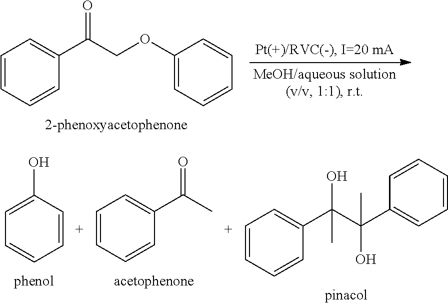

Sample aliquots (250 μL) were taken from the cathode cell during the reaction at timed intervals and diluted with 250 μL of acetonitrile, and the samples were analyzed by HPLC to determine the content of the three reaction products and of the starting material. Yields of each product are reported as a molar % based on the starting amount of 2-phenoxyacetophenone, such that the maximum theoretical yields of phenol and acetophenone are 100%.

For each electroreduction, a current efficiency (CE %) was calculated according to equation (1):

CE%=(mol_{prod}×F×n/Cₜₒₜₐₗ)×100 (1)

where mol_{prod} is the number of moles of reduction products (i.e., phenol and pinacol); F is the Faraday constant (96,485 C/mol); n is the number of electrons per reaction; and Cₜₒₜₐₗ is the total charge passed (in C).

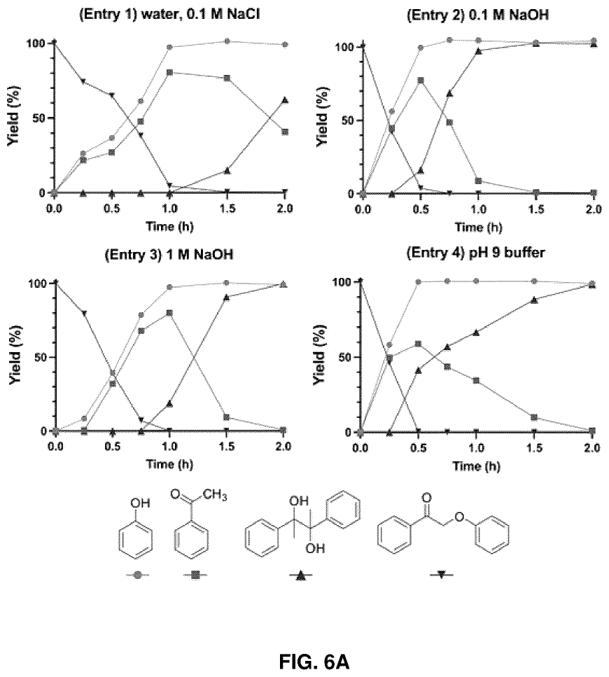

Lignin^{ox} electrolysis. In an H-type electrochemical cell, separated by a Nafion™ 117 membrane and equipped with magnetic stirrers in both compartments, reactions were performed at room temperature with 28 mL of pH 8 phosphate buffer in the anodic cell (platinum wire anode) and 28 mL of a 50/50 (v/v) mixture of 0.1 M aqueous NaOH/methanol in the cathodic cell (RVC cathode, 2 cm² immersed). Sodium chloride (100 mg) was added into the cathodic cell to improve conductivity. Lignin^{ox} (100 mg) was added to the catholyte, and 20 mA current (current density=10 mA/cm²) was applied for 6 h. The mixture was transferred to a 50 mL centrifuge tube, the solvent was removed under nitrogen flow, and the crude product was solubilized in 20 mL of water. The aqueous mixture was acidified with 60% H₂SO₄ to pH 2 to precipitate insoluble residue. The precipitate was filtered, washed with water, and dried in vacuo; this fraction is referred to herein as the “insoluble fraction.” The remaining filtrate was extracted with ethyl acetate (EA; 30 mL×3). The organic layers were combined, dried with anhydrous Na₂SO₄, and evaporated in vacuo; the resulting fraction is referred to herein as the “EA-soluble fraction.” Yields of the EA-soluble and insoluble fractions were determined as a weight percent based on the initial amount of lignin^{ox}. Any difference between the sum of the EA-soluble and insoluble fraction yields and 100% yield was attributed to a third fraction, referred to herein as the “mass balance” fraction); that is, mass balance yield was calculated as 100%−(EA-soluble fraction yield+insoluble fraction yield). The mass balance fraction can be attributed in part to experimental losses as well as formation of water-soluble products of lignin depolymerization. A schematic illustration of the reaction work-up is shown in FIG. 5.

Characterization of Products. DDQ oxidation of the lignins was verified by NMR. Briefly, ¹H¹³C-gradient heteronuclear single quantum coherence (HSQC) NMR was performed at ambient temperature on a 500 MHz Bruker NMR spectrometer equipped with a 5 mm iProbe (BBO probe). Spectra were recorded by utilizing the Bruker pulse sequence “hsqcedetgpsisp2.3” with acquisition times of 63.9 ms (F2, 512 complex points for ¹H) and 63.9 ms (F1, 1024 increments for the ¹³C dimension) using a delay of 1.5 s and 48 scans per increment with spectral widths of 8013 Hz (¹H) and 20 kHz (¹³C). The β-O-4 linkage content of lignins following Kraft, soda, GVL, or Cu-AHP pretreatment was estimated from the HSQC NMR results according to a method described in Das et al., ACS Sustain. Chem. Eng., 2018, 6, 3367-3374, the entire contents of which are hereby incorporated by reference.

Molecular weight profiles, including Mw, Mn, and polydispersity index (i.e., PDI, defined as Mw/Mn), of lignins before and after electrolysis were determined by gel permeation chromatography (GPC). Specifically, acid-insoluble products were analyzed by GPC using a Waters Ultrahydrogel™ 250 7.8×300 mm column equipped with a Waters Ultrahydrogel™ 6×40 mm guard column. Samples for analysis were pre-solubilized in 0.1 M NaOH. Analyses were performed at 40° C. with a mobile phase of 5 mM NaOH in 80/20 (v/v) 0.1 M aqueous sodium nitrate/acetonitrile at a flow rate of 0.7 mL/min. Polystyrene sulfonic acid and poly(ethylene glycol) were employed as external standards.

EA-soluble products resulting from lignin depolymerization were identified and quantified by gas chromatography-mass spectrometry (GC/MS),using a 6890 Agilent GC equipped with a VF-5 ms 30 m×0.25 mm×0.25 μm with a 10 m EZ-Guard column and a 5975b single quadrupole MS detector, using the authentic compounds as standards. Yields are reported based on the original weight of lignin^{ox}.

### Example 1: Electroreductive Cleavage of a Model Compound

The electroreduction of 2-phenoxyacetophenone, as a model compound for lignin, was performed in solvent mixtures comprising 50/50 (v/v) mixtures of methanol and each aqueous solution listed in Table 1. Table 1 also lists the current density used for each electroreduction and the current efficiency measured for each electroreduction.

TABLE 1

| | Current | | Current |
|---|---|---|---|
| | Density | | Efficiency |
| Entry | (mA/cm²) | Aqueous Solution | (%) |
| 1 | 10 | 0.1M NaCl | 48 |
| 2 | 10 | 0.1M NaOH | 35 |
| 3 | 10 | 1M NaOH | 25 |
| 4 | 10 | pH 9 buffer | 33 |
| 5 | 10 | 0.1M NaOH + 0.1M NaCl | 31 |
| 6 | 5 | 0.1M NaOH | 72 |
| 7 | 10 | 0.1M HCl | 15 |
| 8 | 10 | 0.05M H₂SO₄ | 20 |
| 9 | 10 | 0.1M H₂SO₄ | 21 |

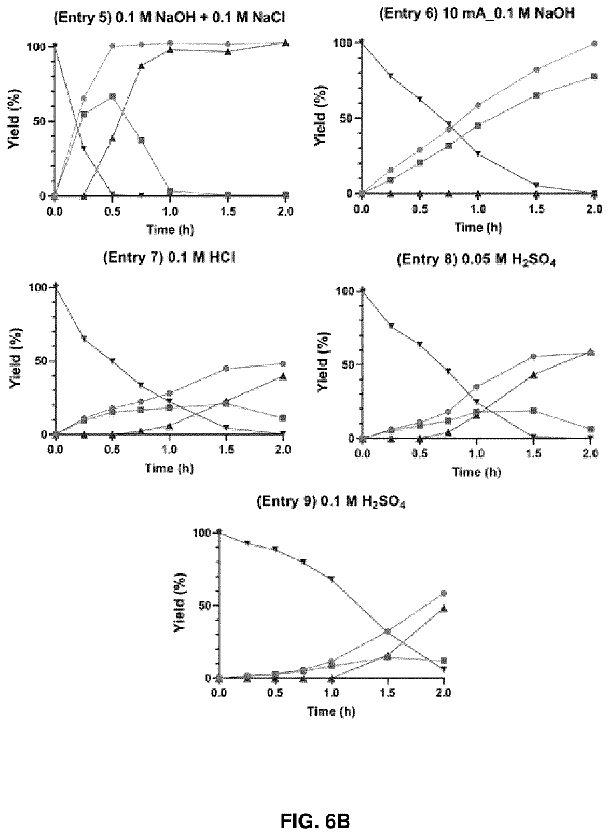

Plots of the in-process yields of phenol, acetophenone, pinacol, and the % consumption of starting material, for the electroreductions corresponding to Entries 1 to 9 are shown in FIGS. 6A and 6B.

Initially, 0.1 M NaCl was used as the aqueous solution (Entry 1); this provided a reference reaction to monitor pH changes during the reaction. Under these conditions, the starting material 2-phenoxyacetophenone was cleaved to phenol (97.3% yield) and acetophenone (80.6% yield) in approximately 1 h, and the pH of the solution stabilized at approximately 13. After 1 h, however, the undesired product pinacol, derived from the further reduction of acetophenone, began to accumulate. As shown in FIG. 6A, the beginning of pinacol formation during the electroreduction corresponding to Entry 1 coincided with the near complete consumption of the starting material.

Under basic conditions (Entry 2, with 0.1 M NaOH as the aqueous solution; reaction pH=13), the reaction provided a >99% yield of phenol and near complete consumption of the substrate within 30 minutes. The acetophenone yield at 30 minutes was 77.3%, though this was accompanied by the formation of pinacol, as shown in FIG. 6A.

Increasing the NaOH concentration from 0.1 M to 1.0 M (Entry 3), buffering the reaction at pH 9 (such as with a phosphate buffer; Entry 4), or employing a combination of 0.1 M NaOH and 0.1 M NaCl (Entry 5) did not substantially alter the reaction profiles, although the yields of pinacol varied. Under reduced current conditions (Entry 6), the electroreduction was slower, but no substantial pinacol formation was detected and high yields of the desired products (>99% phenol, 77.7% acetophenone) were eventually obtained. Performing the reaction under acidic conditions (Entries 7-9) resulted in lower reaction rates, as evidenced by slower consumption of the substrate and slower formation of phenol and acetophenone. The pH of the reactions initiated under acidic conditions stabilized at roughly 13 as protons were converted into H₂. Without intending to be bound by theory, it is believed that the decreased reaction rate under acidic conditions is due to competitive consumption of electrons by proton reduction to generate H₂.

In view of the results for 2-phenoxyacetophenone, the conditions of Entry 2 were selected for depolymerizing the lignin samples. Without intending to be bound by theory, while electroreduction of the model compound at higher current density generated greater amounts of an undesired side product (pinacol, generated by dimerization of acetophenone), the methoxylated aromatic ketones formed by the electrolysis of authentic lignin are expected to be less susceptible to this dimerization. Furthermore, lignins are generally more soluble in basic media than in acidic or neutral media.

### Example 2: Electroreductive Cleavage of Lignins

Initially, electrolysis of raw (i.e., unoxidized) GVL and Cu-AHP lignins was evaluated, using a current density of 10 mA/cm² for 6 h. Table 2 shows the yields of EA-soluble, insoluble, and mass balance fractions from electrolysis of unoxidized GVL and Cu-AHP lignins. As seen in Table 2, electrolysis of the unoxidized lignins provided very low yields of EA-soluble material, indicating low conversion. Accordingly, lignin samples for further evaluation were pre-oxidized using DDQ, as described above.

TABLE 2

| Yield (wt. %) | EA-soluble | Insoluble | Mass Balance |
|---|---|---|---|
| GVL | 1.0 | 93.5 | 5.5 |
| Cu-AHP | 4.6 | 94.0 | 1.4 |

Pre-oxidation of each lignin, in particular the oxidation of β-O-4 linkages, was validated by ¹H¹³C-HSQC NMR, as evidenced by a significant decrease in the correlation peaks associated with the α-OH sites.

The pre-oxidized lignins were electrolyzed at ambient temperature and pressure for 6 h using a current density of 10 mA/cm². A control sample of each pre-oxidized lignin was incubated at ambient temperature and pressure but in the absence of current. The yields of EA-soluble, insoluble, and mass balance fractions from incubation in the absence of current and from electrolysis are shown in Table 3.

TABLE 3

| Yield (wt. %) | | Kraft^{ox} | Soda^{ox} | GVL^{ox} | Cu-AHP^{ox} |
|---|---|---|---|---|---|
| EA-soluble | No current | 4.0 | 3.5 | 5.1 | 6.5 |
| | 10 mA/cm² | 11.8 | 6.5 | 19.4 | 32.0 |
| Insoluble | No current | 95.1 | 96.3 | 90.2 | 92.4 |
| | 10 mA/cm² | 65.3 | 80.2 | 56.2 | 39.0 |
| Mass Balance | No current | 0.9 | 0.2 | 4.7 | 1.1 |
| | 10 mA/cm² | 22.9 | 13.3 | 24.4 | 29.0 |

As seen in Table 3, applying current increased the extent of depolymerization of each of the lignin^{ox} feedstocks; roughly 90-96% of each lignin^{ox} remained inert (i.e., acid-insoluble) following incubation in the absence of current. The yield of EA-soluble products increased from 3.5-6.5% for the lignins that were incubated in the absence of current to 6.5-32.0% for the lignins that were treated with a current density of 10 mA/cm². The increase in yield of EA-soluble products was concomitant with a decrease in the yield of insoluble lignin residue. Electrolysis of Cu-AHP^{ox} afforded the highest content of EA-soluble products (32.0%) and the lowest insoluble fraction (39.0%) among the four oxidized lignins; GVL^{ox} afforded 19.4% and 56.2% of the EA-soluble and insoluble fractions, respectively. Kraft^{ox} and Soda^{ox} showed slight increases in EA-soluble products upon electrolysis. Without intending to be bound by theory, it is believed that the Kraft^{ox} and Soda^{ox} generally have higher levels of crosslinking than GVL^{ox} and Cu-AHP^{ox} and that the higher levels of crosslinking resulted in less efficient depolymerization of Kraft^{ox} and Soda^{ox} compared to GVL^{ox} and Cu-AHP^{ox}.

Results of GC/MS analysis of the compositions of the EA-soluble fractions are shown in Table 4. The amounts of five depolymerization products (vanillin, p-OH benzoic acid, acetosyringone, propiosyringone, and syringic acid, the structures of which are shown in Table 5) that were identified in the EA-soluble fractions of one or more of the lignin samples are shown; “others” indicates the amount of EA-soluble species that were not identified by GC/MS.

TABLE 4

| Yield (wt. %) | Kraft^{ox} | Soda^{ox} | GVL^{ox} | Cu-AHP^{ox} |
|---|---|---|---|---|
| EA-soluble | 11.8 | 6.5 | 19.4 | 32.0 |
| Vanillin | | | 1.1 | 1.9 |
| p-OH-benzoic acid | | | 6.8 | 4.3 |
| Acetosyringone | | 1.4 | 1.4 | 2.4 |
| Propiosyringone | | | 4.3 | 5.0 |
| Syringic acid | 3.6 | 3.4 | 4.0 | 5.8 |
| Others | 8.2 | 1.8 | 1.8 | 12.7 |
| Acid-Insoluble | 65.3 | 80.2 | 56.2 | 39.0 |
| Mass Balance | 22.9 | 13.3 | 24.4 | 29.0 |

TABLE 5

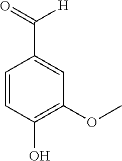

Vanillin

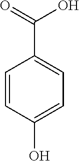

p-OH benzoic acid

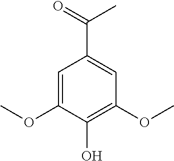

Acetoosyringone

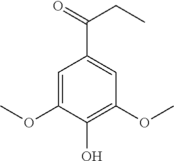

Propiosyringo

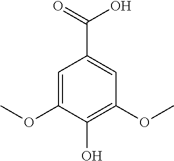

syringic acid

In general, yields of the depolymerization products from electrolysis of GVL^{ox} and Cu-AHP^{ox} were higher than yields from Kraft^{ox} and Soda^{ox}, indicating a greater extent of lignin depolymerization for GVL^{ox} and Cu-AHP^{ox}. Electrolysis of GVL^{ox} and Cu-AHP^{ox} lignins afforded significantly higher monomer yields, forming vanillin, p-OH benzoic acid, acetosyringone, propiosyringone, and syringic acid as the major products, collectively accounting for 17.6 wt. % and 19.4 wt. %, respectively, of the isolated material, based on the initial weight of the oxidized lignin.

The μ-O-4 contents of lignin samples following each of the pretreatments, as estimated from HSQC NMR, are shown in Table 6. In general, Kraft and soda pretreatments employ harsher conditions (e.g., ˜175° C., pH>10) than GVL and Cu-AHP pretreatments. Without intending to be bound by theory, it is believed that Kraft and soda pretreatments introduce more crosslinking compared to GVL and Cu-AHP pretreatments, as evidenced by lower β-O-4 linkage content following Kraft or soda pretreatment. It is further believed that lower crosslinking and higher β-O-4 linkage content in GVL^{ox} and Cu-AHP^{ox} lignins makes these lignins more susceptible to depolymerization by electrochemical reduction than Kraft^{ox} and Soda^{ox} lignins, providing higher yields of EA-soluble products, as shown in Table 4.

TABLE 6

| Lignin | β-O-4 content |
|---|---|
| Kraft | 11% |
| Soda | 21% |
| GVL | 46% |
| Cu-AHP | 59% |

As a follow-up experiment, the insoluble Cu-AHP lignin residue remaining after the electrolysis treatment was further treated with 10 mA/cm² of current density for an additional 6 h. Following this treatment, only 4.78 wt. % of additional EA-soluble products were isolated. Thus, prolonged electrolysis time did not significantly enhance depolymerization yield.

GPC analysis provided additional evidence of the limited cleavage of Kraft^{ox} and Soda^{ox} lignin following electrolytic treatment. Table 7 shows Mw and Mn (in Da) and PDI for the oxidized lignins prior to electrolytic treatment and for the acid-insoluble products (“Residue”) following electrolytic treatment. Mw and PDI of the Kraft^{ox} and Soda^{ox} lignins decreased slightly following electrolytic treatment, while Mw and PDI of the GVL^{ox} and Cu-AHP^{ox} lignins were more significantly reduced following electrolytic treatment. In particular, electrolytic treatment reduced the Mw of Cu-AHP^{ox} from approximately 25,000 Da to 6,200 Da and decreased the PDI from 5.1 to 2.2, indicating depolymerization of high-molecular weight lignin material upon electrolysis.

TABLE 7

| | | Kraft^{ox} | | Soda^{ox} | | GVL^{ox} | | Cu-AHP^{ox} |
|---|---|---|---|---|---|---|---|---|
| | | Residue | Soda^{ox} | Residue | GVL^{ox} | Residue | Cu-AHP^{ox} | Residue |
| Mw | 106,000 | 90,000 | 82,000 | 79,000 | 30,000 | 16,000 | 25,000 | 6,200 |
| Mn | 24,000 | 25,000 | 18,000 | 21,000 | 5,300 | 4,700 | 4,900 | 2,800 |
| PDI | 4.5 | 3.6 | 4.6 | 3.8 | 5.7 | 3.4 | 5.1 | 2.2 |

Summary: Relative to conventional chemical lignin deconstruction approaches, simple reductive electrolysis as disclosed herein provides an alternative, low-waste method for ether cleavage and monomer release. Advantages of this process include the following: (1) reactions can be powered by renewable electricity sources and technologies; (2) generation of H2 and O2 from water splitting could potentially be employed in multiple reactions simultaneously; (3) compared to reagent-based reactions, isolation of products and catalysts is simplified (unless mediators are required in the system) as electrodes are easily separated from products by physical removal (e.g., even in cases where one or both electrodes serves as a heterogeneous catalytic electrode); and (4) reactions can be easily monitored as they do not require special pressure vessels.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

Throughout the specification, where the compounds, compositions, methods, and processes are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

1. A method for depolymerizing lignin, the method comprising:
oxidizing a lignin compound to form an oxidized lignin compound; and
electrochemically reducing the oxidized lignin compound to depolymerize the oxidized lignin compound and to form a depolymerized lignin product having a reduced molecular weight relative to the lignin compound prior to electrochemical reduction.

2. The method of claim 1, wherein:
oxidizing the lignin compound comprises performing a chemical oxidation on the lignin compound.

3. The method of claim 1, wherein:
oxidizing the lignin compound comprises performing anodic oxidation.

4. The method of claim 1, wherein the lignin compound comprises an isolated lignin obtained from one or more biomass pretreatment methods selected from the group consisting of a γ-valerolactone/sulfuric acid (GVL) pretreatment and a copper-catalyzed alkaline hydrogen peroxide (Cu-AHP) pretreatment.

5. The method of claim 1, wherein the lignin compound comprises a phenolic polymer comprising at least one of β-O-4 ether linkages, α-O-4 ether linkages, 4-O-5 ether linkages, β-β carbon-carbon linkages, β-5 carbon-carbon linkages, and 5-5 carbon-carbon linkages between phenolic monomer units in the phenolic polymer.

6. The method of claim 5, wherein the depolymerized lignin product has 50% or less linkages between phenolic monomer units in the phenolic polymer relative to the lignin compound prior to electrochemical reduction.

7. The method of claim 1, wherein the depolymerized lignin product has a number- or weight-average molecular weight of 50% or less relative to the number- or weight-average molecular weight of the lignin compound prior to electrochemical reduction.

8. The method of claim 1, wherein the lignin compound prior to electrochemical reduction has a number-average molecular weight of at least 400 g/mol.

9. The method of claim 8, wherein the depolymerized lignin product has a number-average molecular weight of 50% or less relative to the number-average molecular weight of the lignin compound prior to electrochemical reduction.

10. The method of claim 1, wherein the lignin compound prior to reacting has a weight-average molecular weight of at least 10000 g/mol.

11. The method of claim 10, wherein the depolymerized lignin product has a weight-average molecular weight of 50% or less relative to the weight-average molecular weight of the lignin compound prior to electrochemical reduction.

12. The method of claim 1, wherein the depolymerized lignin product comprises at least one of a lignin monomer unit and a lignin oligomer thereof, the lignin monomer unit comprising an aromatic group with one or more substituents selected from the group consisting of alcohol groups, ether groups, aldehyde groups, ketone groups, alkyl groups, carboxyl groups, and combinations thereof.

13. The method of claim 12, wherein the lignin monomer unit is selected from the group consisting of syringic acid, vanillin, p-OH benzoic acid, acetosyringone, propiosyringone, phenol, cresol, guaiacol, 4-ethyl-guaiacol, eugenol, isoeugenol, methoxyeugenol, syringol, and combinations thereof.

14. The method of claim 12, wherein the depolymerized lignin product comprises lignin monomer units in an amount of 5 wt. % to 50 wt. % relative to the lignin compound prior to electrochemical reduction.

15. The method of claim 1, wherein:
(i) an ethyl acetate-soluble fraction of the depolymerized lignin product constitutes 5 wt. % to 50 wt. % (or 15-50 wt. %) of the depolymerized lignin product;
(ii) a water-soluble fraction of the depolymerized lignin product constitutes 5 wt. % to 50 wt. % (or 15-50 wt. %) of the depolymerized lignin product; and/or
(iii) combined ethyl acetate- and water-soluble fractions of the depolymerized lignin product constitute 10 wt. % to 80 wt. % (or 30-80 wt. %) of the depolymerized lignin product.

16. The method of claim 1, comprising electrochemically reducing the oxidized lignin compound in an aqueous electrolyte medium.

17. The method of claim 16, wherein the aqueous electrolyte medium comprises:
water;
optionally a solvent selected from the group consisting of methanol, ethanol, n-propanol, iso-propanol, n-butanol, iso-butanol, tert-butanol, acetic acid, formic acid, and combinations thereof; and
an electrolyte;
wherein the aqueous electrolyte medium has a pH value of at least 8.

18. The method of claim 16, wherein the oxidized lignin compound is initially present the aqueous electrolyte medium in an amount of 0.1 g/L to 100 g/L.

19. The method of claim 1, comprising electrochemically reducing the oxidized lignin compound in a cathode compartment of a divided electrochemical cell.

20. The method of claim 1, comprising electrochemically reducing the oxidized lignin compound in an undivided electrochemical cell.

21. The method of claim 1, comprising electrochemically reducing the oxidized lignin compound:
(i) at a temperature in a range from 20° C. to 200° C.;
(ii) at a pressure in a range of 0.5 bar to 5 bar;
(iii) for a time in a range of 0.1 hr to 50 hr; and/or
(iv) at a current density in a range of 1 mA/cm² to 1000 mA/cm².

22. The method of claim 1, comprising electrochemically reducing the oxidized lignin compound in the absence of an added thio compound.

23. A method for depolymerizing lignin, the method comprising:
providing an oxidized lignin compound; and
electrochemically reducing the oxidized lignin compound to depolymerize the oxidized lignin compound and to form a depolymerized lignin product having a reduced molecular weight relative to the lignin compound prior to electrochemical reduction,
wherein the oxidized lignin compound comprises a water- or acid-soluble lignin fraction obtained from one or more biomass pretreatment methods.

24.-42. (canceled)
